(19) United States
(12) Patent Application Publication
(10) Pub. No.: US 2026/0256518 A1
(43) Pub. Date: Sep. 3, 2026

(54) OPTICAL ANALYZER ASSEMBLY AND METHOD FOR INTRAVASCULAR LITHOTRIPSY DEVICE

(71) Applicant: Boston Scientific Scimed Inc., Maple Grove, MN (US)

(72) Inventors: Christopher A. Cook, Laguna Niguel, CA (US); Eric Schultheis, San Clemente, CA (US)

(73) Assignee: Boston Scientific Scimed Inc., Maple Grove, MN (US)

(21) Appl. No.: 19/650,492

(22) Filed: Apr. 17, 2026

Related U.S. Application Data
(63) Continuation of application No. 17/172,980, filed on Feb. 10, 2021, now Pat. No. 12,611,253.
(60) Provisional application No. 62/991,394, filed on Mar. 18, 2020.

(51) Int. Cl. A61B 18/24; A61B 18/00; A61B 18/04; A61B 18/20; A61B 18/22; A61B 18/26

(52) U.S. Cl. CPC A61B 18/245; A61B 18/042; A61B 18/26; A61B 2018/0022; A61B 2018/00369; A61B 2018/00386; A61B 2018/2015; A61B 2018/2035; A61B 2018/20361; A61B 2018/204; A61B 2018/2288

(57) ABSTRACT

A catheter system for treating a treatment site within or adjacent to a vessel wall includes a light source, a balloon, a light guide, and an optical analyzer assembly. The light source generates light energy. The balloon is positionable substantially adjacent to the vascular lesion. The balloon has a balloon wall that defines a balloon interior that receives a balloon fluid. The light guide receives light energy from the light source at a guide proximal end and guides the light energy toward a guide distal end and into the balloon interior. The optical analyzer assembly is configured to optically analyze light energy emitted from the guide proximal end of the light guide.

### RELATED APPLICATION

This application is a continuation of U.S. Non-Provisional application Ser. No. 17/172,980, filed on Feb. 10, 2021, now U.S. Pat. No. 12,611,253, issued Apr. 28, 2026, which claims priority to U.S. Provisional Application No. 62/991,394, filed on Mar. 18, 2020. As far as permitted, the contents of U.S. Non-Provisional application Ser. No. 17/172,980 and U.S. Provisional Application No. 62/991,394 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

### SUMMARY

The present invention is directed toward a catheter system for treating a treatment site within or adjacent to a vessel wall or a heart valve. In various embodiments, the catheter system includes a light source, a balloon, a light guide and an optical analyzer assembly. The light source generates light energy. The balloon is positionable substantially adjacent to the treatment site. The balloon has a balloon wall that defines a balloon interior that receives a balloon fluid. The light guide is configured to receive the light energy at a guide proximal end and guide the light energy in a first direction from the guide proximal end toward a guide distal end that is positioned within the balloon interior. The optical analyzer assembly is configured to optically analyze light energy from the light guide that moves in a second direction opposite from the first direction.

In some embodiments, the balloon fluid is provided to the balloon interior so that the balloon expands from a collapsed configuration to an expanded configuration.

Additionally, in certain embodiments, the light source generates pulses of light energy that are guided along the light guide into the balloon interior to induce plasma generation in the balloon fluid within the balloon interior. In some such embodiments, the catheter system further includes a plasma generator that is positioned at the guide distal end of the light guide, the plasma generator being configured to generate plasma in the balloon fluid within the balloon interior. Further, in such embodiments, the plasma generation can cause rapid bubble formation and impart pressure waves upon the balloon wall adjacent to the vascular lesion.

In such embodiments, the optical analyzer assembly can be configured to optically detect whether plasma generation has occurred in the balloon fluid within the balloon interior. Additionally, the optical analyzer assembly can further be configured to optically detect whether a lack of plasma generation has occurred in the balloon fluid within the balloon interior. Further, the optical analyzer assembly can also be configured to optically detect a failure of the light guide at any point along a length of the light guide from the guide proximal end to the guide distal end. In certain such embodiments, the optical analyzer assembly can also be configured to optically detect potential damage to the light guide at any point along a length of the light guide from the guide proximal end to the guide distal end. Moreover, in some such embodiments, the optical analyzer assembly is configured to automatically shut down operation of the catheter system upon optical detection of potential damage to the light guide.

In some embodiments, the guide distal end includes a distal light receiver that receives light energy through the light guide from the guide distal end to the guide proximal end as a returning energy beam. In certain such embodiments, the light energy that is received by the light guide from the guide distal end to the guide proximal end is emitted from the plasma that is generated in the balloon fluid within the balloon interior. Further, in some such embodiments, the light energy that is received by the light guide from the guide distal end to the guide proximal end via the distal light receiver is optically analyzed by the optical analyzer assembly.

In certain embodiments, the catheter system further includes a pulse generator that is coupled to the light source. The pulse generator is configured to trigger the light source to emit pulses of light energy that are guided along the light guide from the guide proximal end to the guide distal end. In such embodiments, the pulses of light energy can energize a plasma generator that is positioned at the guide distal end of the light guide, the plasma generator being configured to generate plasma in the balloon fluid within the balloon interior. Additionally, in certain such embodiments, light energy is guided back through the light guide to the guide proximal end as a returning energy beam. In such embodiments, the optical analyzer assembly is configured to optically analyze the returning energy beam to determine whether plasma generation has occurred in the balloon fluid within the balloon interior.

In some embodiments, the optical analyzer assembly includes a beamsplitter and a photodetector. The beamsplitter is configured to receive the returning energy beam and direct at least a portion of the returning energy beam onto the photodetector. Additionally, in certain embodiments, the catheter system further includes an optical element that is positioned along a beam path between the beamsplitter and the photodetector, the optical element being configured to couple the at least a portion of the returning energy beam onto the photodetector. Further, in some embodiments, the photodetector generates a signal based at least in part on visible light that is included with the at least a portion of the returning energy beam. Additionally, the signal from the photodetector can be amplified with an amplifier to provide an amplified signal, and the amplified signal can be directed to control electronics to determine an intensity of the plasma generation in the balloon fluid within the balloon interior. Still further, in some embodiments, the amplified signal is gated using a discriminator circuit. In such embodiments, the control electronics compare timing of the pulse of energy from the light source as triggered by the pulse generator with the timing of the amplified signal from the photodetector to determine when plasma generation occurred in the balloon fluid within the balloon interior.

Additionally, in other embodiments, the catheter system further includes a second light source that generates light energy as an interrogation beam. In such embodiments, the light guide is configured to receive the interrogation beam from the second light source at the guide proximal end and guide the interrogation beam from the second light source toward the guide distal end. In some such embodiments, the catheter system further includes a pulse generator that is coupled to the second light source, the pulse generator being configured to trigger the second light source to emit pulses of light energy as interrogation beams that are guided along the light guide from the guide proximal end to the guide distal end. Additionally, in certain such embodiments, the second light source is a visible light source.

Further, in certain embodiments, the catheter system further includes a plasma generator that is positioned at the guide distal end of the light guide. In such embodiments, the interrogation beam is one of scattered by and reflected by the plasma generator and is directed along the light guide from the guide distal end to the guide proximal end as a returned interrogation beam. In certain embodiments, the returned interrogation beam is optically analyzed by the optical analyzer assembly as emitted from the guide proximal end of the light guide. Additionally, in some embodiments, the optical analyzer assembly includes a beamsplitter and a photodetector, and the beamsplitter in configured to receive the returned interrogation beam and direct at least a portion of the returned interrogation beam onto the photodetector. Further, in certain such embodiments, the photodetector generates a signal based at least in part on the at least a portion of the returned interrogation beam. Additionally, the signal from the photodetector can be amplified with an amplifier to provide an amplified signal; and the amplified signal can be directed to control electronics to determine when plasma generation occurred in the balloon fluid within the balloon interior. Still further, the amplified signal can be gated using a discriminator circuit. In such embodiments, the control electronics can compare timing of the pulse of light energy from the second light source as triggered by the pulse generator with the timing of the amplified signal from the photodetector to determine when plasma generation occurred in the balloon fluid within the balloon interior.

In some embodiments, the light source includes a laser.

Additionally, in certain embodiments, the light source includes an infrared laser that emits light energy in the form of pulses of infrared light.

Further, in some embodiments, the light guide includes an optical fiber.

In certain applications, the present invention is further directed toward a method for treating a vascular lesion within or adjacent to a vessel wall, the method including the steps of generating light energy with a light source; positioning a balloon substantially adjacent to the vascular lesion, the balloon having a balloon wall that defines a balloon interior that receives a balloon fluid; receiving light energy from the light source with a light guide at a guide proximal end; guiding the light energy with the light guide from the guide proximal end toward a guide distal end and into the balloon interior; and optically analyzing light energy emitted from the guide proximal end of the light guide with an optical analyzer assembly.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of an embodiment of a catheter system in accordance with various embodiments herein, the catheter system including an optical analyzer assembly having features of the present invention;

FIG. 2 is a simplified schematic view of a portion of an embodiment of the catheter system including an embodiment of the optical analyzer assembly; and

FIG. 3 is a simplified schematic view of a portion of another embodiment of the catheter system including another embodiment of the optical analyzer assembly.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions (also sometimes referred to herein as “treatment sites”) can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

The catheter systems and related methods disclosed herein are configured to monitor the performance, reliability and safety of an intravascular lithotripsy (IVL) catheter. In various embodiments, the catheter systems of the present invention utilize an energy source, e.g., a light source such as a laser source or another suitable energy source, which provides energy that is guided by an energy guide, e.g., a light guide, to create a localized plasma in a balloon fluid within a balloon interior of an inflatable balloon of the catheter. As such, the energy guide can sometimes be referred to herein as, or can be said to incorporate a “plasma generator” at or near a guide distal end of the energy guide that is positioned within the balloon interior. This localized plasma induces pressure waves that impart pressure onto and induce fractures in a treatment site within or adjacent to a blood vessel wall within a body of a patient. As used herein, the treatment site can include a vascular lesion such as a calcified vascular lesion or a fibrous vascular lesion, typically found in a blood vessel and/or a heart valve.

In particular, in various embodiments, the catheter systems can include a catheter configured to advance to the treatment site within or adjacent a blood vessel or heart valve within the body of the patient. The catheter includes a catheter shaft, and a balloon that is coupled and/or secured to the catheter shaft. The balloons herein can include a balloon wall that defines the balloon interior and can be configured to receive the balloon fluid within the balloon interior to expand from a collapsed configuration suitable for advancing the catheter through a patient's vasculature, to an expanded configuration suitable for anchoring the catheter in position relative to the treatment site. The catheter systems also include one or more energy guides, e.g., light guides, disposed along the catheter shaft and within the balloon. Each energy guide can be configured for generating pressure waves within the balloon for disrupting the vascular lesions. The catheter systems utilize energy from an energy source, e.g., light energy from a light source, to generate the plasma, i.e. via the plasma generator, within the balloon fluid at or near a guide distal end of the energy guide disposed in the balloon located at the treatment site. The plasma formation can initiate one or more pressure waves and can initiate the rapid formation of one or more bubbles that can rapidly expand to a maximum size and then dissipate through a cavitation event that can launch pressure waves upon collapse. The rapid expansion of the plasma-induced bubbles can generate one or more pressure waves within the balloon fluid retained within the balloon and thereby impart pressure waves upon the treatment site. In some embodiments, the energy source can be configured to provide sub-millisecond pulses of energy, e.g., light energy, from the energy source to initiate plasma formation in the balloon fluid within the balloon to cause rapid bubble formation and to impart pressure waves upon the balloon wall at the treatment site. Thus, the pressure waves can transfer mechanical energy through an incompressible balloon fluid to the treatment site to impart a fracture force on the treatment site.

Importantly, as described in detail herein, the catheter systems of the present invention include an optical analyzer assembly that is configured to provide real-time continuous monitoring of the light emitted from the light guide into the balloon interior, which can be used to detect that a plasma event has occurred, and can also be used as a monitor for nominal operation of the catheter system. Additionally, the optical analyzer assembly can also be utilized to measure the intensity of the light energy emitted from the light guide in order to provide an accurate measurement of the energy output of the plasma generator that is incorporated as part of the light guide. More specifically, the measurement of the energy output of the plasma generator can be used in conjunction with the known energy input from the energy source to determine the conversion efficiency. Such metric can also be used to assess the condition of the plasma generator and light guide and determine if the catheter system is performing normally, as well as the number of operation cycles remaining.

More specifically, in various embodiments, as described in detail herein, the present invention comprises a means of sampling light returned from the plasma generator and/or from the balloon interior back through the light guide. It is appreciated that light energy can travel in both, opposing directions along the length of the light guide. Thus, it is possible to detect light originating at the guide distal end of the light guide, or at any other position along the length of the light guide, at a guide proximal end of the light guide. Such light energy that is transmitted back through the light guide will thus be separated and detected and/or analyzed via the optical analyzer assembly to effectively monitor the performance, reliability and safety of the catheter system as described in detail herein.

It is appreciated that the continuous monitoring of the light energy emitted from the plasma generator, and the measuring of the intensity of the emitted light energy, through use of the present invention, as described in detail herein, addresses multiple potential issues with the performance, reliability and safety of an IVL catheter, in particular one that utilizes an energy source to create a localized plasma which in turn produces a high energy bubble inside a balloon catheter. Specific issues this invention addresses include: 1) optical detection of successful firing of the energy source, e.g., the laser source, to generate the plasma within the balloon interior, 2) accurate determination of the energy output of the plasma generator, 3) optical detection of a failure of the catheter system to generate the desired plasma within the balloon interior, and 4) optical detection of a failure of the light guide at any point along the length of the light guide.

As used herein, the terms “intravascular lesion”, “vascular lesion” and “treatment site” are used interchangeably unless otherwise noted. As such, the intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

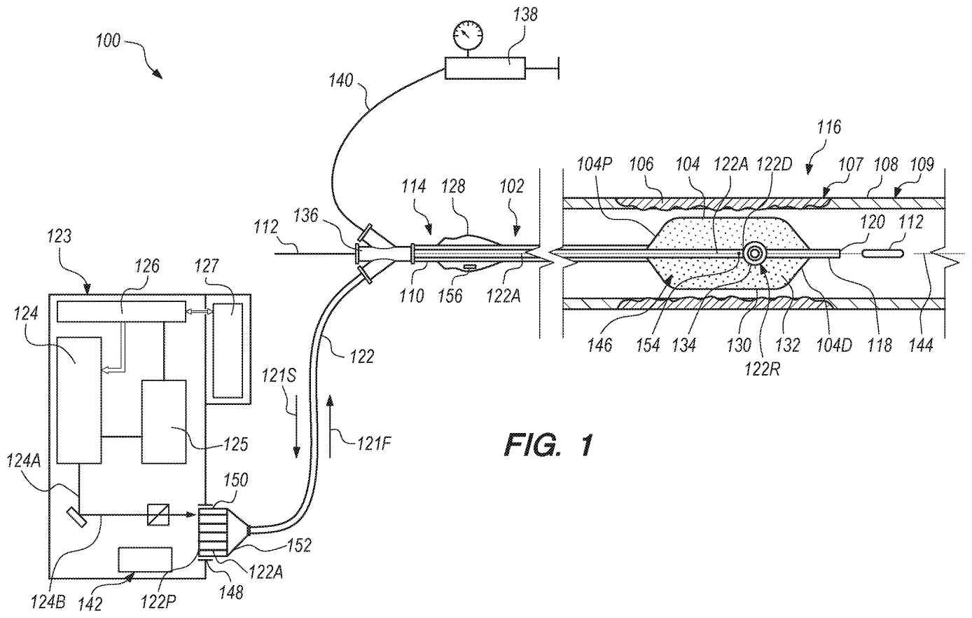

lt is appreciated that the catheter systems disclosed herein can include many different forms. Referring now to FIG. 1, a schematic cross-sectional view is shown of a catheter system 100 in accordance with various embodiments herein. As described herein, the catheter system 100 is suitable for imparting pressure to induce fractures in one or more vascular lesions within or adjacent a vessel wall of a blood vessel, or on or adjacent to a heart valve within a body of a patient. In the embodiment illustrated in FIG. 1, the catheter system 100 can include one or more of a catheter 102, a light guide bundle 122 including one or more light guides 122A, a source manifold 136, a fluid pump 138, a system console 123 including one or more of a light source 124, a power source 125, a system controller 126, and a graphic user interface 127 (a “GUI”), a handle assembly 128, and an optical analyzer assembly 142.

The catheter 102 is configured to move to a treatment site 106 within or adjacent to a blood vessel 108 within a body 107 of a patient 109. The treatment site 106 can include one or more vascular lesions such as calcified vascular lesions, for example. Additionally, or in the alternative, the treatment site 106 can include vascular lesions such as fibrous vascular lesions.

The catheter 102 can include an inflatable balloon 104 (sometimes referred to herein simply as a “balloon”), a catheter shaft 110 and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter shaft 110 can also include a guidewire lumen 118 which is configured to move over the guidewire 112. The catheter shaft 110 can further include an inflation lumen (not shown). In some embodiments, the catheter 102 can have a distal end opening 120 and can accommodate and be tracked over the guidewire 112 as the catheter 102 is moved and positioned at or near the treatment site 106.

The catheter shaft 110 of the catheter 102 can be coupled to the one or more light guides 122A of the light guide bundle 122 that are in optical communication with the light source 124. The light guide(s) 122A can be disposed along the catheter shaft 110 and within the balloon 104. In some embodiments, each light guide 122A can be an optical fiber and the light source 124 can be a laser. The light source 124 can be in optical communication with the light guides 122A at the proximal portion 114 of the catheter system 100.

In some embodiments, the catheter shaft 110 can be coupled to multiple light guides 122A such as a first light guide, a second light guide, a third light guide, etc., which can be disposed at any suitable positions about the guidewire lumen 118 and/or the catheter shaft 110. For example, in certain non-exclusive embodiments, two light guides 122A can be spaced apart by approximately 180 degrees about the circumference of the guidewire lumen 118 and/or the catheter shaft 110; three light guides 122A can be spaced apart by approximately 120 degrees about the circumference of the guidewire lumen 118 and/or the catheter shaft 110; or four light guides 122A can be spaced apart by approximately 90 degrees about the circumference of the guidewire lumen 118 and/or the catheter shaft 110. Still alternatively, multiple light guides 122A need not be uniformly spaced apart from one another about the circumference of the guidewire lumen 118 and/or the catheter shaft 110. More particularly, it is further appreciated that the light guides 122A described herein can be disposed uniformly or non-uniformly about the guidewire lumen 118 and/or the catheter shaft 110 to achieve the desired effect in the desired locations.

The balloon 104 can include a balloon wall 130 that defines a balloon interior 146, and can be inflated with a balloon fluid 132 to expand from a collapsed configuration suitable for advancing the catheter 102 through a patient's vasculature, to an expanded configuration suitable for anchoring the catheter 102 in position relative to the treatment site 106. Stated in another manner, when the balloon 104 is in the expanded configuration, the balloon wall 130 of the balloon 104 is configured to be positioned substantially adjacent to the treatment site 106, i.e. to the vascular lesion(s). In some embodiments, the light source 124 of the catheter system 100 can be configured to provide sub-millisecond pulses of light from the light source 124, along the light guides 122A, to a location within the balloon interior 146 of the balloon 104, thereby inducing plasma formation in the balloon fluid 132 within the balloon interior 146 of the balloon 104. The plasma formation causes rapid bubble formation, and imparts pressure waves upon the treatment site 106. Exemplary plasma-induced bubbles are shown as bubbles 134 in FIG. 1.

It is appreciated that although the catheter systems 100 illustrated herein are generally described as including a light source 124 and one or more light guides 122A, the catheter system 100 can alternatively include any suitable energy source and energy guides for purposes of generating the desired plasma in the balloon fluid 132 within the balloon interior 146.

The balloons 104 suitable for use in the catheter systems 100 described in detail herein include those that can be passed through the vasculature of a patient when in the collapsed configuration. In some embodiments, the balloons 104 herein are made from silicone. In other embodiments, the balloons 104 herein are made from polydimethylsiloxane (PDMS), polyurethane, polymers such as PEBAX™ material available from Arkema, which has a location at King of Prussia, Pennsylvania, USA, nylon, and the like. In some embodiments, the balloons 104 can include those having diameters ranging from one millimeter (mm) to 25 mm in diameter. In some embodiments, the balloons 104 can include those having diameters ranging from at least 1.5 mm to 12 mm in diameter. In some embodiments, the balloons 104 can include those having diameters ranging from at least one mm to five mm in diameter.

Additionally, in some embodiments, the balloons 104 herein can include those having a length ranging from at least five mm to 300 mm. More particularly, in some embodiments, the balloons 104 herein can include those having a length ranging from at least eight mm to 200 mm. It is appreciated that balloons 104 of greater length can be positioned adjacent to larger treatment sites 106, and, thus, may be usable for imparting pressure onto and inducing fractures in larger vascular lesions or multiple vascular lesions at precise locations within the treatment site 106.

Further, the balloons 104 herein can be inflated to inflation pressures of between approximately one atmosphere (atm) and 70 atm. In some embodiments, the balloons 104 herein can be inflated to inflation pressures of from at least 20 atm to 70 atm. In other embodiments, the balloons 104 herein can be inflated to inflation pressures of from at least six atm to 20 atm. In still other embodiments, the balloons 104 herein can be inflated to inflation pressures of from at least three atm to 20 atm. In yet other embodiments, the balloons 104 herein can be inflated to inflation pressures of from at least two atm to ten atm.

Still further, the balloons 104 herein can include those having various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloons 104 herein can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The balloon fluid 132 can be a liquid or a gas. Exemplary balloon fluids 132 suitable for use herein can include, but are not limited to one or more of water, saline, contrast medium, fluorocarbons, perfluorocarbons, gases, such as carbon dioxide, and the like. In some embodiments, the balloon fluids 132 described can be used as base inflation fluids. In some embodiments, the balloon fluids 132 include a mixture of saline to contrast medium in a volume ratio of 50:50. In other embodiments, the balloon fluids 132 include a mixture of saline to contrast medium in a volume ratio of 25:75. In still other embodiments, the balloon fluids 132 include a mixture of saline to contrast medium in a volume ratio of 75:25. Additionally, the balloon fluids 132 suitable for use herein can be tailored on the basis of composition, viscosity, and the like in order to manipulate the rate of travel of the pressure waves therein. In certain embodiments, the balloon fluids 132 suitable for use herein are biocompatible. A volume of balloon fluid 132 can be tailored by the chosen light source 124 and the type of balloon fluid 132 used.

In some embodiments, the contrast agents used in the contrast media herein can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

Additionally, the balloon fluids 132 herein can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the balloon fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system. By way of non-limiting examples, various lasers described herein can include neodymium: yttrium-aluminum-garnet (Nd:YAG−emission maximum=1064 nm) lasers, holmium: YAG (Ho:YAG−emission maximum=2.1 μm) lasers, or erbium: YAG (Er:YAG−emission maximum=2.94 μm) lasers. In some embodiments, the absorptive agents used herein can be water soluble. In other embodiments, the absorptive agents used herein are not water soluble. In some embodiments, the absorptive agents used in the balloon fluids 132 herein can be tailored to match the peak emission of the light source 124. Various light sources 124 having emission wavelengths of at least ten nanometers to one millimeter are discussed elsewhere herein.

It is appreciated that the catheter system 100 and/or the light guide bundle 122 disclosed herein can include any number of light guides 122A in optical communication with the light source 124 at the proximal portion 114, and with the balloon fluid 132 within the balloon interior 146 of the balloon 104 at the distal portion 116. For example, in some embodiments, the catheter system 100 and/or the light guide bundle 122 can include from one light guide 122A to five light guides 122A. In other embodiments, the catheter system 100 and/or the light guide bundle 122 can include from five light guides 122A to fifteen light guides 122A. In yet other embodiments, the catheter system 100 and/or the light guide bundle 122 can include from ten light guides 122A to thirty light guides 122A. Alternatively, in still other embodiments, the catheter system 100 and/or the light guide bundle 122 can include greater than 30 light guides 122A.

The light guides 122A herein can include an optical fiber or flexible light pipe. The light guides 122A herein can be thin and flexible and can allow light signals to be sent with very little loss of strength. The light guides 122A herein can include a core surrounded by a cladding about its circumference. In some embodiments, the core can be a cylindrical core or a partially cylindrical core. The core and cladding of the light guides 122A can be formed from one or more materials, including but not limited to one or more types of glass, silica, or one or more polymers. The light guides 122A may also include a protective coating, such as a polymer. It is appreciated that the index of refraction of the core will be greater than the index of refraction of the cladding.

Each light guide 122A can guide light along its length from a proximal portion, i.e. a guide proximal end 122P, to a distal portion, i.e. a guide distal end 122D, having at least one optical window (not shown) that is positioned within the balloon interior 146. The light guides 122A can create a light path as a portion of an optical network including the light source 124. The light path within the optical network allows light to travel from one part of the network to another. Both the optical fiber and the flexible light pipe can provide a light path within the optical networks herein.

As provided herein, the guide distal end 122D can further include and/or incorporate a distal light receiver 122R that enables light energy to be moved back into and through the light guide 122A from the guide distal end 122D to the guide proximal end 122P. Stated another way, the light energy can move in a first direction 121F along the light guide 122A that is generally from the guide proximal end 122P toward the guide distal end 122D of the light guide 122A. At least a portion of the light energy can also move in a second direction 121S along the light guide 122A that is substantially opposite the first direction 121F, i.e. from the guide distal end 122D toward the guide proximal end 122P of the light guide 122A. Moreover, as described in greater detail herein below, the light energy emitted from the guide proximal end 122P after being moved back through the light guide 122A (in the second direction 121S) can be separated and then optically detected, interrogated and/or analyzed through use of the optical analyzer assembly 142.

Further, the light guides 122A herein can assume many configurations about and/or relative to the catheter shaft 110 of the catheters 102 described herein. In some embodiments, the light guides 122A can run parallel to the longitudinal axis 144 of the catheter shaft 110. In some embodiments, the light guides 122A can be physically coupled to the catheter shaft 110. In other embodiments, the light guides 122A can be disposed along a length of an outer diameter of the catheter shaft 110. In yet other embodiments, the light guides 122A herein can be disposed within one or more light guide lumens within the catheter shaft 110.

Additionally, it is further appreciated that the light guides 122A can be disposed at any suitable positions about the circumference of the guidewire lumen 118 and/or the catheter shaft 110, and the guide distal end 122D of each of the light guides 122A can be disposed at any suitable longitudinal position relative to the length of the balloon 104 and/or relative to the length of the guidewire lumen 118.

Further, the light guides 122A herein can include one or more photoacoustic transducers 154, where each photoacoustic transducer 154 can be in optical communication with the light guide 122A within which it is disposed. In some embodiments, the photoacoustic transducers 154 can be in optical communication with the guide distal end 122D of the light guide 122A. Additionally, in such embodiments, the photoacoustic transducers 154 can have a shape that corresponds with and/or conforms to the guide distal end 122D of the light guide 122A.

The photoacoustic transducer 154 is configured to convert light energy into an acoustic wave at or near the guide distal end 122D of the light guide 122A. It is appreciated that the direction of the acoustic wave can be tailored by changing an angle of the guide distal end 122D of the light guide 122A.

It is further appreciated that the photoacoustic transducers 154 disposed at the guide distal end 122D of the light guide 122A herein can assume the same shape as the guide distal end 122D of the light guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. It is also appreciated that the light guide 122A can further include additional photoacoustic transducers 154 disposed along one or more side surfaces of the length of the light guide 122A.

The light guides 122A described herein can further include one or more diverting features or “diverters” (not shown in FIG. 1) within the light guide 122A that are configured to direct light to exit the light guide 122A toward a side surface e.g., at or near the guide distal end 122D of the light guide 122A, and toward the balloon wall 130. A diverting feature can include any feature of the system herein that diverts light from the light guide 122A away from its axial path toward a side surface of the light guide 122A. Additionally, the light guides 122A can each include one or more light windows disposed along the longitudinal or axial surfaces of each light guide 122A and in optical communication with a diverting feature. Stated in another manner, the diverting features herein can be configured to direct light in the light guide 122A toward a side surface, e.g., at or near the guide distal end 122D, where the side surface is in optical communication with a light window. The light windows can include a portion of the light guide 122A that allows light to exit the light guide 122A from within the light guide 122A, such as a portion of the light guide 122A lacking a cladding material on or about the light guide 122A.

Examples of the diverting features suitable for use herein include a reflecting element, a refracting element, and a fiber diffuser. Additionally, the diverting features suitable for focusing light away from the tip of the light guides 122A herein can include, but are not to be limited to, those having a convex surface, a gradient-index (GRIN) lens, and a mirror focus lens. Upon contact with the diverting feature, the light is diverted within the light guide 122A to the photoacoustic transducer 154 that is in optical communication with a side surface of the light guide 122A. As noted, the photoacoustic transducer 154 then converts light energy into an acoustic wave that extends away from the side surface of the light guide 122A.

The source manifold 136 can be positioned at or near the proximal portion 114 of the catheter system 100. The source manifold 136 can include one or more proximal end openings that can receive the plurality of light guides 122A of the light guide bundle 122, the guidewire 112, and/or an inflation conduit 140 that is coupled in fluid communication with the fluid pump 138. The catheter system 100 can also include the fluid pump 138 that is configured to inflate the balloon 104 with the balloon fluid 132 as needed.

As noted above, in the embodiment illustrated in FIG. 1, the system console 123 includes one or more of the light source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the light source 124, the power source 125, the system controller 126, and the GUI 127 can be provided within the catheter system 100 without the specific need for the system console 123.

Further, as illustrated in FIG. 1, in certain embodiments, at least a portion of the optical analyzer assembly 142 can also be positioned substantially within the system console 123. Alternatively, components of the optical analyzer assembly 142 can be positioned in a different manner than what is specifically shown in FIG. 1.

Additionally, as shown, the system console 123, and the components included therewith, is operatively coupled to the catheter 102, the light guide bundle 122, and the remainder of the catheter system 100. For example, in some embodiments, as illustrated in FIG. 1, the system console 123 can include a console connection aperture 148 (also sometimes referred to generally as a “socket”) by which the light guide bundle 122 is mechanically coupled to the system console 123. In such embodiments, the light guide bundle 122 can include a guide coupling housing 150 (also sometimes referred to generally as a “ferrule”) that houses a portion, e.g., the guide proximal end 122P, of each of the light guides 122A. The guide coupling housing 150 is configured to fit and be selectively retained within the console connection aperture 148 to provide the desired mechanical coupling between the light guide bundle 122 and the system console 123.

Further, the light guide bundle 122 can also include a guide bundler 152 (or “shell”) that brings each of the individual light guides 122A closer together so that the light guides 122A and/or the light guide bundle 122 can be in a more compact form as it extends with the catheter 102 into the blood vessel 108 during use of the catheter system 100.

As provided herein, the light source 124 can be selectively and/or alternatively coupled in optical communication with each of the light guides 122A, i.e. to the guide proximal end 122P of each of the light guides 122A, in the light guide bundle 122. In particular, the light source 124 is configured to generate light energy in the form of a source beam 124A, e.g., a pulsed source beam, that can be selectively and/or alternatively directed to and received by each of the light guides 122A in the light guide bundle 122 as an individual guide beam 124B. Alternatively, the catheter system 100 can include more than one light source 124. For example, in one non-exclusive alternative embodiment, the catheter system 100 can include a separate light source 124 for each of the light guides 122A in the light guide bundle 122.

The light source 124 can have any suitable design. In certain embodiments, as noted above, the light source 124 can be configured to provide sub-millisecond pulses of light from the light source 124 that are focused onto a small spot in order to couple it into the guide proximal end 122P of the light guide 122A. Such pulses of light energy are then directed along the light guides 122A to a location within the balloon 104, thereby inducing plasma formation in the balloon fluid 132 within the balloon interior 146 of the balloon 104. In particular, the light energy emitted at the guide distal end 122D of the light guide 122A energizes the plasma generator to form the plasma within the balloon fluid 132 within the balloon interior 146. The plasma formation causes rapid bubble formation, and imparts pressure waves upon the treatment site 106. In such embodiments, the sub-millisecond pulses of light from the light source 124 can be delivered to the treatment site 106 at a frequency of between approximately one hertz (Hz) and 5000 Hz. In some embodiments, the sub-millisecond pulses of light from the light source 124 can be delivered to the treatment site 106 at a frequency of between approximately 30 Hz and 1000 Hz. In other embodiments, the sub-millisecond pulses of light from the light source 124 can be delivered to the treatment site 106 at a frequency of between approximately ten Hz and 100 Hz. In yet other embodiments, the sub-millisecond pulses of light from the light source 124 can be delivered to the treatment site 106 at a frequency of between approximately one Hz and 30 Hz. Alternatively, the sub-millisecond pulses of light can be delivered to the treatment site 106 at a frequency that can be greater than 5000 Hz.

It is appreciated that although the light source 124 is typically utilized to provide pulses of light energy, the light source 124 can still be described as providing a single source beam 124A, i.e. a single pulsed source beam.

The light sources 124 suitable for use herein can include various types of light sources including lasers and lamps. For example, in certain non-exclusive embodiments, the light source 124 can be an infrared laser that emits light energy in the form of pulses of infrared light. Alternatively, as noted above, the light sources 124, as referred to herein, can include any suitable type of energy source.

Suitable lasers can include short pulse lasers on the sub-millisecond timescale. In some embodiments, the light source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the balloon fluid 132 of the catheters 102 described herein. In various embodiments, the pulse widths can include those falling within a range including from at least ten ns to 200 ns. In some embodiments, the pulse widths can include those falling within a range including from at least 20 ns to 100 ns. In other embodiments, the pulse widths can include those falling within a range including from at least one ns to 500 ns.

Additionally, exemplary nanosecond lasers can include those within the UV to IR spectrum, spanning wavelengths of about ten nanometers (nm) to one millimeter (mm). In some embodiments, the light sources 124 suitable for use in the catheter systems 100 herein can include those capable of producing light at wavelengths of from at least 750 nm to 2000 nm. In other embodiments, the light sources 124 can include those capable of producing light at wavelengths of from at least 700 nm to 3000 nm. In still other embodiments, the light sources 124 can include those capable of producing light at wavelengths of from at least 100 nm to ten micrometers (μm). Nanosecond lasers can include those having repetition rates of up to 200 kHz. In some embodiments, the laser can include a Q-switched thulium: yttrium-aluminum-garnet (Tm:YAG) laser. In other embodiments, the laser can include a neodymium: yttrium-aluminum-garnet (Nd:YAG) laser, holmium: yttrium-aluminum-garnet (Ho:YAG) laser, erbium: yttrium-aluminum-garnet (Er:YAG) laser, excimer laser, helium-neon laser, carbon dioxide laser, as well as doped, pulsed, fiber lasers.

The catheter systems 100 disclosed herein can generate pressure waves having maximum pressures in the range of at least one megapascal (MPa) to 100 MPa. The maximum pressure generated by a particular catheter system 100 will depend on the light source 124, the absorbing material, the bubble expansion, the propagation medium, the balloon material, and other factors. In some embodiments, the catheter systems 100 herein can generate pressure waves having maximum pressures in the range of at least two MPa to 50 MPa. In other embodiments, the catheter systems 100 herein can generate pressure waves having maximum pressures in the range of at least two MPa to 30 MPa. In yet other embodiments, the catheter systems 100 herein can generate pressure waves having maximum pressures in the range of at least 15 MPa to 25 MPa.

The pressure waves described herein can be imparted upon the treatment site 106 from a distance within a range from at least 0.1 millimeters (mm) to 25 mm extending radially from the light guides 122A when the catheter 102 is placed at the treatment site 106. In some embodiments, the pressure waves can be imparted upon the treatment site 106 from a distance within a range from at least ten mm to 20 mm extending radially from the light guides 122A when the catheter 102 is placed at the treatment site 106. In other embodiments, the pressure waves can be imparted upon the treatment site 106 from a distance within a range from at least one mm to ten mm extending radially from the light guides 122A when the catheter 102 is placed at the treatment site 106. In yet other embodiments, the pressure waves can be imparted upon the treatment site 106 from a distance within a range from at least 1.5 mm to four mm extending radially from the light guides 122A when the catheter 102 is placed at the treatment site 106. In some embodiments, the pressure waves can be imparted upon the treatment site 106 from a range of at least two MPa to 30 MPa at a distance from 0.1 mm to ten mm. In some embodiments, the pressure waves can be imparted upon the treatment site 106 from a range of at least two MPa to 25 MPa at a distance from 0.1 mm to ten mm.

The power source 125 is electrically coupled to and is configured to provide necessary power to each of the light source 124, the system controller 126, the GUI 127, the handle assembly 128, and the optical analyzer assembly 142. The power source 125 can have any suitable design for such purposes.

As noted, the system controller 126 is electrically coupled to and receives power from the power source 125. Additionally, the system controller 126 is coupled to and is configured to control operation of each of the light source 124, the GUI 127 and the optical analyzer assembly 142. The system controller 126 can include one or more processors or circuits for purposes of controlling the operation of at least the light source 124, the GUI 127 and the optical analyzer assembly 142. For example, the system controller 126 can control the light source 124 for generating pulses of light energy as desired, e.g., at any desired firing rate. Additionally, the system controller 126 can control and/or operate in conjunction with the optical analyzer assembly 142 to effectively provide real-time continuous monitoring of the performance, reliability and safety of the catheter system 100.

Additionally, the system controller 126 can further be configured to control operation of other components of the catheter system 100, e.g., the positioning of the catheter 102 adjacent to the treatment site 106, the inflation of the balloon 104 with the balloon fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. Additionally, the GUI 127 is electrically connected to the system controller 126. With such design, the GUI 127 can be used by the user or operator to ensure that the catheter system 100 is employed as desired to impart pressure onto and induce fractures into the vascular lesions at the treatment site 106. Additionally, the GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time, e.g., during use of the catheter system 100. Further, in various embodiments, the GUI 127 can include one or more colors, different sizes, varying brightness, etc., that may act as alerts to the user or operator. Additionally, or in the alternative, the GUI 127 can provide audio data or information to the user or operator. It is appreciated that the specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. Additionally, in this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the light source 124, the fluid pump 138, the GUI 127 and the optical analyzer assembly 142. In some embodiments, the handle assembly 128 can integrate and/or include at least a portion of the system controller 126 within an interior of the handle assembly 128. For example, as shown, in certain such embodiments, the handle assembly 128 can include circuitry 156 that can form at least a portion of the system controller 126. Additionally, in some embodiments, the circuitry 156 can receive electrical signals or data from the optical analyzer assembly 142. Further, or in the alternative, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, e.g., within the system console 123. It is understood that the handle assembly 128 can include fewer or additional components than those specifically illustrated and described herein.

As an overview, and as provided in greater detail herein, the optical analyzer assembly 142 is configured to effectively monitor the performance, reliability and safety of the catheter system 100. During use of the catheter system 100, when the plasma initially forms in the balloon fluid 132 within the balloon interior, the plasma emits broad-spectrum electromagnetic radiation. Additionally, as noted above, at least a portion of the light energy emitted can reflect off of, or otherwise be received by, the distal light receiver 122R near the guide distal end 122D of the light guide 122A. Such portion of the light energy can thus travel back through the light guide 122A in the second direction 121S to the guide proximal end 122P where it can be separated and detected. The intensity and timing of the visible light pulse relative to the plasma-generating pulse from the light source 124 provides an indication that the plasma generator functioned, its energy output, and its functional condition. It is appreciated that visible light flashes may occur in other locations along the length of the light guide 122A if the light guide 122A is damaged or broken. Such additional light flashes will also be coupled into the light guide 122A and carried back in the second direction 121S to the guide proximal end 122P. The intensity and timing of these additional light pulses can indicate a damaged light guide 122A or plasma generator.

It is appreciated that the failure of an energy-driven plasma generator or associated light guide 122A, e.g., if the light guide 122A breaks or is damaged during the use of the catheter system 100, could lead to patient or operator harm resulting from the leaked energy. Potential harms include tissue burns and retinal damage. As noted above, in some embodiments, the energy source 124 is a laser that emits invisible infrared light, making visible detection by the operator impossible. Thus, if the optical analyzer assembly 142 indicates any such failures to have occurred, the procedure and energy delivery, e.g., laser energy delivery, must be stopped immediately to mitigate the associated risks to the patient and the operator. Stated in another manner, with the design of the optical analyzer assembly 142 described herein, the present invention detects any noted failures within the catheter system 100, e.g., breaking of, damage to, or failure of the light guide 122A and/or the plasma generator, and provides an indicator or signal that the system controller 126 can use to lock out the energy source 124. This provides a necessary safety interlock for a potentially hazardous condition in which the energy source 124 can leak out in an undesirable way. Moreover, the system controller 126 could be used to indicate to the surgeon, e.g., via the GUI 127, to halt the procedure and remove the catheter 102 from the patient 109 under treatment.

Additionally, it is further appreciated that the optical analyzer assembly 142 can have any suitable design for purposes of effectively monitoring the performance, reliability and safety of the catheter system 100. Certain non-exclusive examples of potential designs for the optical analyzer assembly 142 are described in detail herein below.

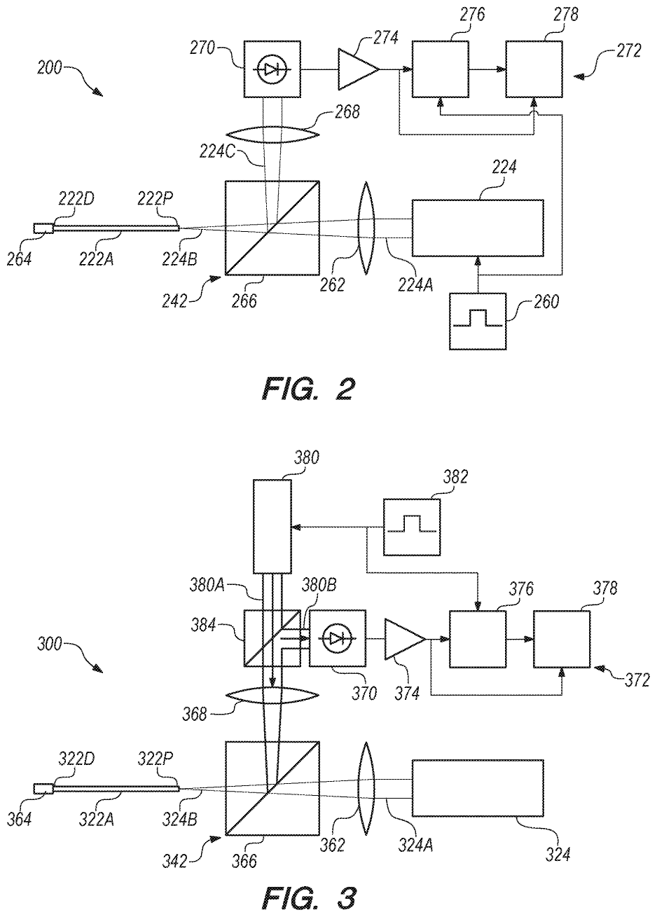

FIG. 2 is a simplified schematic view of a portion of an embodiment of the catheter system 200 including an embodiment of the optical analyzer assembly 242. The design of the catheter system 200 is substantially similar to the embodiments illustrated and described herein above. It is appreciated that various components of the catheter system 200, such as are shown in FIG. 1, are not illustrated in FIG. 2 for purposes of clarity and ease of illustration. However, it is appreciated that the catheter system 200 will likely include most, if not all, of such components.

As shown in FIG. 2, the catheter system 200 again includes an energy source 224 that is configured to generate light energy in the form of a source beam 224A, e.g., a pulsed source beam, that can be selectively and/or alternatively directed to and received by each light guide 222A (only one light guide is illustrated in FIG. 2) as an individual guide beam 224B. In one non-exclusive embodiment, the energy source 224 is an infrared laser source, and the light guide 222A is a small diameter, multimode optical fiber. In the embodiment illustrated in FIG. 2, a pulse generator 260 is coupled to the energy source 224. The pulse generator 260 is configured to trigger the energy source 224, which, thus, emits an energy pulse as the source beam 224A. In certain embodiments, the source beam 224A from the energy source 224 passes through an optical element 262, e.g., a focusing lens, that is configured to focus the source beam 224A as the individual guide beam 224B down onto a guide proximal end 222P of the light guide 222A, thereby coupling the pulse of infrared energy, i.e. the individual guide beam 224B, into the light guide 222A.

Subsequently, the pulse of infrared energy, i.e. the individual guide beam 224B, travels along and/or through the light guide 222A and energizes a plasma generator 264 that is positioned and/or incorporated at or near a guide distal end 222D of the light guide 222A. The plasma generator 264 utilizes the pulse of infrared energy to create a localized plasma in the balloon fluid 132 (illustrated in FIG. 1) within the balloon interior 146 (illustrated in FIG. 1) of the balloon 104 (illustrated in FIG. 1).

Upon creation of the plasma in the balloon fluid 132 within the balloon interior 146, in various embodiments, a pulse of broad-spectrum light energy emitted from the plasma is coupled back into the guide distal end 222D of the light guide 222A. Such pulse of broad-spectrum light energy then travels back along and/or through the light guide 222A from where it is emitted from the guide proximal end 222P of the light guide 222A, i.e. as a returning energy beam 224C.

As described in detail herein, the optical analyzer assembly 242 is configured to effectively monitor the performance, reliability and safety of the catheter system 200 by optically analyzing the light energy emitted from the guide proximal end 222P of the light guide 222A, e.g., the returning energy beam 224C. The design of the optical analyzer assembly 242 can be varied to suit the specific requirements of the catheter system 200. In particular, in the embodiment shown in FIG. 2, the optical analyzer assembly 242 includes one or more of a beamsplitter 266, an optical element 268, e.g., a coupling lens, a photodetector 270, and a signal conditioning and processing system 272. Additionally, as shown, the signal conditioning and processing system 272 can include one or more of an amplifier 274, a discriminator 276, and control electronics 278, which can include one or more processors or circuits. Alternatively, in other embodiments, the optical analyzer assembly 242 and/or the signal conditioning and processing system 272 can include more components or fewer components than what is specifically illustrated and described herein.

As shown, the beamsplitter 266, e.g., a dichroic beamsplitter, is positioned in the optical path of the energy source 224 and the guide proximal end 222P of the light guide 222A. In certain embodiments, the beamsplitter 266 is configured to pass light for wavelengths longer than those visible to the photodetector 270. This can be referred to as the cutoff wavelength. The beamsplitter 266 is further configured to reflect all light having a wavelength that is shorter than the cutoff wavelength. As illustrated in FIG. 2, the returning energy beam 224C that is emitted from the guide proximal end 222P of the light guide 222A is reflected off of the beamsplitter 266 and is coupled into the photodetector 270 using the optical element 268. More particularly, the optical element 268, e.g., a coupling lens, is positioned in the optical path of the returning energy beam 224C after it is reflected off of the beamsplitter 266, between the beamsplitter 266 and the photodetector 270. The optical element 268 effectively images the guide proximal end 222P of the light guide onto the photodetector 270, thereby coupling light energy emitted from the guide proximal end 222P of the light guide 222A, i.e. in the form of the returning energy beam 224C, onto the photodetector 270. With such design, the visible light emitted from the plasma formed at the guide distal end 222D of the light guide 222A is collected by the photodetector 270.

Additionally, in some embodiments, the photodetector 270 generates a signal that is based on the visible light emitted from the plasma formed at the guide distal end 222D of the light guide 222A that has been collected by the photodetector 270. As shown in FIG. 2, the signal from the photodetector 270 is then directed to the signal conditioning and processing system 272, where detection of and intensity evaluation of the plasma event are determined. In particular, in certain embodiments, the signal from the photodetector 270 is directed toward the amplifier 274 where the signal from the photodetector 270 is amplified. The amplified signal is thus utilized, e.g., within the control electronics 278, to determine the intensity of the plasma event that occurred in the balloon fluid 132 within the balloon interior 146.

Further, in certain embodiments, the pulse from the amplified photodetector signal is gated using the discriminator 276, e.g., a discriminator circuit, that is triggered by the pulse from the pulse generator 260. This information can then be used, e.g., within the control electronics 278, to determine when the plasma event occurred in the balloon fluid 132 within the balloon interior 146. More specifically, the control electronics 278 can compare the timing of the original pulse of energy from the energy source 224, as triggered by the pulse generator 260, with the timing of the amplified photodetector signal, as gated using the discriminator 276, to determine when the plasma event occurred in the balloon fluid 132 within the balloon interior 146.

In some embodiments, the control electronics 278 of the signal conditioning and processing system 272 can be included as part of the system controller 126 (illustrated in FIG. 1). Alternatively, the control electronics 278 of the signal conditioning and processing system 272 can be provided independently of the system controller 126 and can be in electrical communication with the system controller 126.

It is appreciated that there are numerous other configurations for the photodetector 270 and the signal conditioning and processing system 272 that are needed to detect and analyze the light pulse returning from the light guide 222A, i.e. the returning energy beam 224C. For example, in another embodiment, the photodetector 270 can be a spectrometer that provides intensity and wavelength information about the returning energy beam 224C. In such embodiment, this information can be used to generate a spectral signature to further identify specific conditions or events in the light guide 222A and/or the plasma generator 264. More particularly, the small quantities of material comprising the plasma generator 264 will be vaporized during its regular operation. These will produce a spectral line that would be distinct. It is further appreciated that this approach could further be used to differentiate between a functioning plasma generator 264 and a broken or damaged light guide 222A.

As described in detail herein, the primary mechanism for the present invention is direct detection of the light pulse created by the plasma event in the balloon fluid 132 within the balloon interior 146. The signal conditioning and processing system 278 can be utilized to indicate the intensity of the light pulse, its spectrum, and when it occurs relative to the input pulse from the energy source 224. This can be interpreted as follows:
1) The light pulse must occur after a time interval determined by the length of the light guide 222A and the duration of the input energy pulse from the energy source 224. If the detected light pulse has the correct intensity and occurs within a specific time window, it is an indication that the plasma generator 264 functioned correctly.
2) If no light pulse is detected at all, it is an indication of device failure.
3) If a smaller light pulse is detected that occurs too early relative to the energy pulse from the energy source 224, this would be an indication of a failure of the light guide 222A.
4) If the light pulse is detected as having a different spectrum or missing a spectral line or signature, this could be used to indicate a device failure.

FIG. 3 is a simplified schematic view of a portion of another embodiment of the catheter system 300 including another embodiment of the optical analyzer assembly 342. The design of the catheter system 300 is substantially similar to the embodiments illustrated and described herein above. It is appreciated that various components of the catheter system 300, such as are shown in FIG. 1, are not illustrated in FIG. 3 for purposes of clarity and ease of illustration. However, it is appreciated that the catheter system 300 will likely include most, if not all, of such components.

As shown in FIG. 3, the catheter system 300 again includes an energy source 324 that is configured to generate light energy in the form of a source beam 324A, e.g., a pulsed source beam, that can be selectively and/or alternatively directed to and received by each light guide 322A (only one light guide is illustrated in FIG. 3) as an individual guide beam 324B. In one non-exclusive embodiment, the energy source 324 is an infrared laser source, and the light guide 322A is a small diameter, multimode optical fiber. In certain embodiments, the energy source 324 can again be configured to provide sub-millisecond pulses of energy as the source beam 324A, which are then focused, e.g., with an optical element 362, onto a small spot in order to couple it as the individual guide beam 324B into the guide proximal end 322P of the light guide 322A.

Subsequently, the individual guide beam 324B travels along and/or through the light guide 322A and energizes a plasma generator 364 that is positioned and/or incorporated at or near a guide distal end 322D of the light guide 322A. The plasma generator 364 utilizes the pulse of infrared energy to create a localized plasma in the balloon fluid 132 (illustrated in FIG. 1) within the balloon interior 146 (illustrated in FIG. 1) of the balloon 104 (illustrated in FIG. 1).

As described in detail herein, the optical analyzer assembly 342 is again configured to effectively monitor the performance, reliability and safety of the catheter system 300, e.g., the light guide 322A and the plasma generator 364, through optical analysis of light energy emitted from the guide proximal end 322P of the light guide 322A. However, in the embodiment illustrated in FIG. 3, the optical analyzer assembly 342 has a different design than in the previous embodiments. More specifically, in this embodiment, rather than detecting and analyzing the light pulse emitted from the plasma or broken section of the light guide as the returning energy beam 224C (illustrated in FIG. 2), a separate, second energy source 380, e.g., a second light source, is used to interrogate the light guide 322A. This approach has similarities to Optical Time Domain Reflectometry (OTDR) which is used for detecting failures in long optical fiber transmission lines.

In particular, in the embodiment shown in FIG. 3, the optical analyzer assembly 342 includes one or more of the second energy source 380, a pulse generator 382, a beamsplitter 366, an optical element 368, e.g., a coupling lens, a second beamsplitter 384, a photodetector 370, and a signal conditioning and processing system 372. Additionally, as shown, the signal conditioning and processing system 372 can include one or more of an amplifier 374, a discriminator 376, and control electronics 378, which can include one or more processors or circuits. Alternatively, in other embodiments, the optical analyzer assembly 342 and/or the signal conditioning and processing system 372 can include more components or fewer components than what is specifically illustrated and described herein.

As shown in the embodiment illustrated in FIG. 3, the pulse generator 382 is coupled to the second energy source 380, with the pulse generator 382 being configured to trigger the second energy source 380, which, thus, emits an energy pulse as an interrogation beam 380A. In one non-exclusive embodiment, the second energy source 380 is a high-intensity, visible wavelength laser, and the pulse generator 382 is used to create a short, high-intensity pulse from the second energy source 380. The interrogation beam 380A is initially directed toward the second beamsplitter 384, which, as described herein, can be used to create separate source and return paths for the second energy source 380. In one embodiment, the second beamsplitter 384 is an ordinary beamsplitter that has a high reflection-to-transmission ratio. This allows a small, but sufficient amount of light energy to be coupled into the light guide 322A.

Additionally, in certain embodiments, the interrogation beam 380A from the second energy source 380 then passes through the optical element 368, and is redirected onto the guide proximal end 322P of the light guide 322A by the beamsplitter 366, e.g., a dichroic beamsplitter. The interrogation beam 380A then travels along and/or through the length of the light guide 322A. The interrogation beam 380A will be scattered or reflected by the plasma generator 364 at or near the guide distal end 322D of the light guide 322A and return to the guide proximal end 322P. The same optical path is then used to collect and detect the returned light pulse, i.e. a returned interrogation beam 380B.

As shown in FIG. 3, the returned interrogation beam 380B is optically analyzed using the optical analyzer assembly 342. More particularly, as shown, the beamsplitter 366 and the optical element 368 are again utilized to separate the light energy returning through the light guide 322A, i.e. the returned interrogation beam 380B, to be emitted from the guide proximal end 322P of the light guide 322A. Subsequently, the returned interrogation beam 380B is directed toward the second beamsplitter 384. As noted above, the second beamsplitter 384 can have a high reflection-to-transmission ratio, which allows collection and detection of a weak reflected pulse from the light guide 322A in the form of the returned interrogation beam 380B. Thus, the portion of the returned interrogation beam 380B that is reflected by the second beamsplitter 384 can be collected and coupled into the photodetector 370. With such design, the optical element 368 effectively images the guide proximal end 322P of the light guide onto the photodetector 370, thereby coupling light energy emitted from the guide proximal end 322P of the light guide 322A, i.e. in the form of the returned interrogation beam 380B, onto the photodetector 370.

Additionally, in some embodiments, the photodetector 370 generates a signal that is based on the portion of the returned interrogation beam 380B that has been collected by the photodetector 370. As shown in FIG. 3, the signal from the photodetector 370 is then directed to the signal conditioning and processing system 372, where detection of the plasma event is determined. In certain embodiments, the signal from the photodetector 370 is directed toward the amplifier 374 where the signal from the photodetector 370 is amplified. Further, in some embodiments, the pulse from the amplified photodetector signal is gated using the discriminator 276, e.g., a discriminator circuit, that is triggered by the pulse from the pulse generator 382. This information can then be used, e.g., within the control electronics 378, to determine when and if the plasma event occurred in the balloon fluid 132 within the balloon interior 146. More specifically, the control electronics 378 can compare the timing of the original pulse of energy from the second energy source 380, as triggered by the pulse generator 382, with the timing of the electronic pulse of the amplified photodetector signal, as gated using the discriminator 376, to indicate where along the light guide 322A the interrogating pulse was returned, i.e. as the returned interrogation beam 380B. This could be conditioned to determine whether the returned interrogation beam 380B was from the plasma generator 364, which would be a maximum time difference between trigger pulse and return pulse. Conversely, a shorter time interval between the trigger pulse and the return pulse would indicate the return was nearer to the guide proximal end 322P of the light guide 322A, which would indicate a failure or break in the light guide.

In some embodiments, the control electronics 378 of the signal conditioning and processing system 372 can be included as part of the system controller 126 (illustrated in FIG. 1). Alternatively, the control electronics 378 of the signal conditioning and processing system 372 can be provided independently of the system controller 126 and can be in electrical communication with the system controller 126.

As noted above, the optical analyzer assembly of the present invention addresses multiple potential issues with the performance, reliability and safety of an IVL catheter, in particular one that utilizes an energy source, e.g., a light source such as a laser source, to create a localized plasma which in turn induces a high energy bubble in the balloon fluid within the balloon interior of the balloon. For example, as noted above, issues that are addressed by the present invention include, but are not limited to: (1) optical detection of successful firing of the energy source and/or the plasma generator to generate the plasma within the balloon interior, (2) accurate determination of the energy output of the plasma generator, (3) optical detection of failure of the catheter system, e.g., the plasma generator, to generate the desired plasma within the balloon interior, and (4) optical detection of a failure of the light guide within the plasma generator, the balloon or along any section of the catheter shaft.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

1. (canceled)

2. A catheter system for treating a treatment site within or adjacent to a vessel wall or a heart valve, the catheter system comprising:
an expandable member positionable adjacent to the treatment site, the expandable member defining an interior region;
one or more light guides configured to receive first light energy from a light source at a guide proximal end and guide the first light energy in a first direction toward a guide distal end positioned within the interior region of the expandable member, the first light energy being configured to generate plasma within the interior region, the one or more light guides further being configured to guide, in a second direction opposite the first direction, second light energy from the guide distal end to the guide proximal end, wherein the second light energy comprises a portion of light energy emitted from the plasma; and
an optical analyzer assembly configured to optically analyze the second light energy received at the guide proximal end that has traveled through a light guide of the one or more light guides in the second direction.

3. The catheter system of claim 2, wherein the optical analyzer assembly determines whether plasma generation has occurred within the interior region.

4. The catheter system of claim 2, wherein the optical analyzer assembly includes a photodetector.

5. The catheter system of claim 4, wherein the optical analyzer assembly further includes a beamsplitter that directs at least a portion of the second light energy toward the photodetector.

6. The catheter system of claim 5, wherein the optical analyzer assembly further includes an optical element positioned between the beamsplitter and the photodetector.

7. The catheter system of claim 2, wherein the optical analyzer assembly determines whether the light guide is damaged.

8. The catheter system of claim 7, wherein the catheter system is configured to cease operation of the light source in response to determining that the light guide is damaged.

9. The catheter system of claim 2, wherein the optical analyzer assembly spectrally analyzes the second light energy.

10. The catheter system of claim 2, wherein the optical analyzer assembly is configured to determine wavelength information associated with the portion of the light energy emitted from the plasma.

11. The catheter system of claim 2, wherein the optical analyzer assembly includes a spectrometer configured to analyze a spectral distribution of the portion of the light energy emitted from the plasma.

12. The catheter system of claim 2, wherein the optical analyzer assembly is configured to identify a plasma generation event based at least in part on spectral information corresponding to the second light energy.

13. The catheter system of claim 2, wherein the optical analyzer assembly is configured to analyze the portion of the light energy emitted from the plasma to determine whether plasma generation occurred.

14. The catheter system of claim 13, wherein the optical analyzer assembly is configured to determine whether plasma generation occurred based at least in part on optical information associated with the portion of the light energy emitted from the plasma.

15. The catheter system of claim 14, wherein the optical information includes beam wavelength information, beam intensity information, or both wavelength information and beam intensity information.

16. A catheter system for treating a treatment site within or adjacent to a vessel wall or a heart valve, the catheter system comprising:
an expandable member defining an interior region;
an energy source configured to generate pulses of energy for generating plasma within the interior region;
one or more light guides configured to guide light energy from a guide distal end of the one or more light guides to a guide proximal end of the one or more light guides, wherein the light energy comprises a portion of light energy emitted from the plasma within the interior region; and
an optical analyzer assembly configured to analyze the light energy to determine whether a plasma event occurred within the interior region.

17. The catheter system of claim 16, wherein the optical analyzer assembly includes a spectrometer configured to analyze wavelength information associated with the light energy.

18. The catheter system of claim 16, wherein the optical analyzer assembly is configured to distinguish between the plasma event and a light guide failure condition.

19. A catheter system for treating a treatment site within or adjacent to a vessel wall or a heart valve, the catheter system comprising:
a light guide configured to transmit light energy toward a plasma generator positioned within an interior region of an expandable member;
a second light source configured to transmit an interrogation beam through the light guide in a first direction; and
an optical analyzer assembly configured to analyze a return interrogation beam returned through the light guide in a second direction different from the first direction, and wherein the analysis of the return interrogation beam determines whether damage has occurred to the light guide.

20. The catheter system of claim 19, wherein the optical analyzer assembly is configured to compare the return interrogation beam to an expected interrogation beam to determine whether the light guide has experienced damage.

21. The catheter system of claim 19, wherein the optical analyzer assembly is configured to determine a location of damage along the light guide.
